(19) United States
(12) Plant Patent Application Publication
(10) Pub. No.: US 2014/0053304 P1
(43) Pub. Date: Feb. 20, 2014

(54) Dahlia plant named 'KLEDH11031'

(50) ;

(71) Applicants: Nils Klemm, Stuttgart (DE); Andrea Dohm, Pforzheim (DE)

(72) Inventors: Nils Klemm, Stuttgart (DE); Andrea Dohm, Pforzheim (DE)

(73) Assignee: KLEMM+SOHN GMBH & CO.KG, Stuttgart (DE)

(21) Appl. No.: 13/573,047

(22) Filed: Aug. 16, 2012

(51) Int. Cl. A01H 5/00

(52) U.S. Cl. PLT/321

(57) ABSTRACT

A new and distinct dahlia variety named ‘KLEDH11031’ particularly distinguished by its large, red-purple flowers, and a full, compact plant habit, is disclosed.

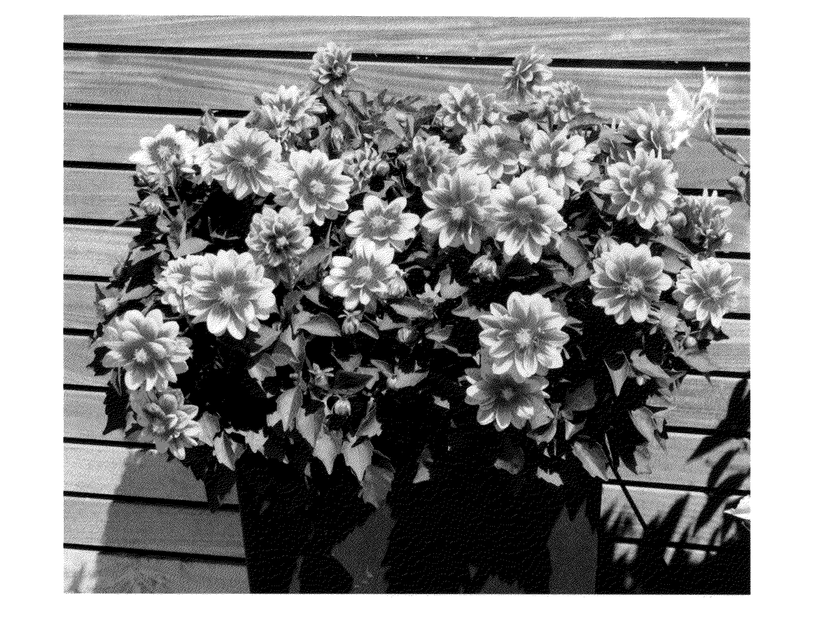

### GENUS AND SPECIES

Dahlia×hybrida

### VARIETY DENOMINATION

‘KLEDH11031’

### BACKGROUND OF THE NEW PLANT

The present invention comprises a new and distinct variety of dahlia, botanically known as Dahlia×hybrida, and hereinafter referred to by the variety name ‘KLEDH11031’. The new variety is the result of a cross conducted in 2007 in Stuttgart, Germany between female dahlia plant ‘DH 2006 0095’ (unpatented) and male dahlia plant ‘DH 2006 0039’ (unpatented). A single plant selection was subsequently chosen for further evaluation and asexual propagation.

The new variety was first asexually propagated by vegetative cuttings in May 2008 in Stuttgart, Germany and has been asexually reproduced repeatedly by terminal tip cuttings for 4 to 5 generations. The present invention has been found to retain its distinctive characteristics through successive asexual propagations.

Plant Breeder's Rights for this variety were applied for in the European Union on Jul. 8, 2011. ‘KLEDH11031’ has not been made publicly available more than one year prior to filing of this application.

### SUMMARY OF THE INVENTION

The following are the most outstanding and distinguishing characteristics of this new variety when grown under normal horticultural practices in Stuttgart, Germany.

1. Large, red-purple flowers; and

2. A full, compact plant habit.

### DESCRIPTION OF THE PHOTOGRAPHS

This new dahlia plant is illustrated by the accompanying photograph which shows overall plant habit including blooms, buds, and foliage of the plant. The photograph is of a four month-old plant grown in a heated greenhouse and outside in Stuttgart, Germany in 2012. The colors shown are as true as can be reasonably obtained by conventional photographic procedures.

### DESCRIPTION OF THE NEW VARIETY

The following detailed descriptions set forth the distinctive characteristics of ‘KLEDH11031’. The data which define these characteristics were collected from asexual reproductions carried out in Stuttgart, Germany. The plant history was taken in May of 2012 on 3 month-old plants grown in 14 centimeter pots. The plants were grown in a heated greenhouse and pinched once at week 7. Color readings were taken under natural light. Color references are primarily to the R.H.S. Colour Chart of The Royal Horticultural Society of London (R.H.S.) (2007 edition).

### DESCRIPTION OF THE NEW PLANT

Classification:
   Family.—Asteraceae.
   Botanical name.—Dahlia×hybrida.
   Common name.—Dahlia.
   Denomination.—‘KLEDH11031’.
Parentage:
   Female parent.—‘DH 2006 0095’ (unpatented).
   Male parent.—‘DH 2006 0039’ (unpatented).
Growth:
   Form and habit.—Inverted triangle.
   Growth habit.—Upright.
   Branching habit.—Freely branching.
   Height (from top of soil).—25.0 cm to 35.0 cm.
   Width (including inflorescence).—Approximately 25.0 cm.
   Propagation.—Vegetative cuttings from tips.
   Time to produce a finished flowering plant.—Approximately 9 weeks.
   Time to initiate and develop roots.—8 to 21 days.
   Root description.—Fine fibrous.
   Rooting habit.—Fusiform.
Leaves:
   Arrangement.—Opposite.
   Shape.—Ovate.
   Apex.—Acuminate.
   Base.—Attenuate.
   Margin.—Serrate.
   Immature leaf.—Color: Upper surface: RHS N137A Lower surface: RHS 148C.
   Mature leaf.—Color: Upper surface: RHS N137A Lower surface: RHS 139C.
   Length.—Approximately 11.0 cm.
   Width.—Approximately 7.0 cm.
   Texture (Both upper and lower surface).—Leathery and rough.
   Venation pattern.—Arcuate.
   Petioles.—Length: Approximately 3.5 cm Diameter: Approximately 0.5 cm Color: RHS 138B Texture: Smooth.
Stems:
   Total number of branches.—5.
   Length.—Approximately 26.0 cm.
   Diameter.—0.8 cm.
   Internode length.—Approximately 3.8 cm.
   Color.—RHS 141C.
   Texture.—Smooth.
Inflorescence buds:
   Shape.—Round.
   Length.—1.2 cm.
   Diameter.—1.3 cm.
   Color (at tight bud just before the ray florets unfold).—RHS 144B at the apex and RHS 141B at the base.
Inflorescence:
   Type.—Terminal and composite.
   Blooming habit (flowering season).—Spring to autumn.
   Quantity of inflorescences per plant.—Approximately 5.
   Lastingness of the inflorescences on the plant.—20 days.
   Fragrance.—Absent.
   Inflorescence diameter.—Approximately 8.5 cm.
   Disc diameter.—1.6 cm.
Disc floret:
   Quantity per inflorescence.—30.
   Shape.—Tubular.
   Tube color (closed).—RHS 7A.
   Tube color (mature).—RHS 6A.
   Length.—0.8 cm.
   Diameter (at apex).—0.2 cm.
   Apex.—Five pointed.
   Apex color.—RHS 7A.
   Base.—Fused into a corolla tube.
   Margin.—Entire.
Ray floret:
   Quantity per inflorescence.—Approximately 20.
   Shape.—Ovate.
   Color.—
   Upper surface.—RHS 68B at the apex and RHS 53A at the base Lower surface: RHS 76B at the apex and RHS 33B at the base.
   Length.—3.3 cm.
   Width.—1.8 cm.
   Apex.—Emarginate.
   Base.—Fused into a corolla tube.
   Margin.—Entire.
   Texture.—Smooth.
   Peduncle.—Length: Approximately 5.0 cm Diameter: 0.3 cm Texture: Smooth Color: RHS 143A.
Phyllaries:
   Arrangement.—Whorled.
   Quantity.—Approximately 24.
   Shape.—Lanceolate.
   Color.—Upper surface: RHS 143B Lower surface: RHS 143C.
   Length.—1.6 cm.
   Width.—0.5 cm.
   Apex.—Acute.
   Base.—Obtuse.
   Margin.—Entire.
   Texture.—Smooth.
Reproductive organs:
   Androecium.—Location: Present on the disc floret Stamens: Quantity: Approximately 30 Shape: Narrow elliptic Color: RHS 20A Filament length: 0.25 cm Filament diameter: 0.05 cm Anther: Shape: Narrow elliptic Color: RHS 20A Length: 0.3 cm Diameter: 0.15 cm Pollen: Color: RHS 16A Amount: Moderate.
   Gynoecium.—Location: Present at the base of ray and on the disc florets Pistils: Number: 30 Length: 1.5 cm Diameter: 0.3 cm Stigma: Color: RHS 6A Shape: Bipartite Length: 0.3 cm Diameter: 0.1 cm Style: Color: RHS 13A Length: 0.8 cm Diameter: 0.1 cm Shape: Filamentous.
Fruit and seed set: Has not been observed
Disease and insect resistance: Has not been observed

We claim:

1. A new and distinct variety of dahlia plant named ‘KLEDH11031’ as shown and described herein.
